(19) United States
(12) Patent Application Publication
(10) Pub. No.: US 2013/0006239 A1
(43) Pub. Date: Jan. 3, 2013

(54) ELECTROSURGICAL SYSTEMS AND METHODS

(75) Inventors: John Pikramenos, Astoria, NY (US); Jonathan Achenbach, Kay, TX (US); Albert M. Juergens, Boylston, MA (US); Frank D'Amelio, Los Olivos, CA (US)

(73) Assignee: ELLMAN INTERNATIONAL, INC., Oceanside, NY (US)

(21) Appl. No.: 13/175,618

(22) Filed: Jul. 1, 2011

(51) Int. Cl. A61B 18/14; H01R 43/00; A61N 1/18

(52) U.S. Cl. 606/41; 607/2; 29/825

(57) ABSTRACT

An electrosurgical device can have a housing and an electrode defining an energizable surface at least partially positioned externally of the housing. Such a device can have a first circuit and a second circuit. The housing can define a handpiece. The first circuit can be configured to selectively direct energy to a power element. The power element can be configured to selectively electrically couple to the electrode. The second circuit can have a selectively operable handpiece accessory. The handpiece can also have a device configured to direct suitable energy from the first circuit to the second circuit to power the second circuit. In some embodiments, the second circuit can be configured to selectively interrupt the first circuit. For example, the second circuit can interrupt the first circuit when a cumulative duration of operation of the second circuit exceeds an upper threshold duration. Methods of operating such handpieces are also disclosed.

### BACKGROUND

The innovations and related subject matter disclosed herein (collectively referred to as the “disclosure”) generally pertain to electrosurgical systems, such as electrosurgical devices and related electrical circuitry and methods. More particularly, the innovations relate to electrosurgical systems that use a first circuit in an electrosurgical device to power an electrode and a second circuit in the device to power an accessory system, the second circuit scavenging power from the first circuit.

FIG. 1 shows a typical electrosurgical system having a control unit 34 and an electrosurgical device 10. The electrosurgical device 10 includes a housing 12, e.g., for circuitry, and an energizable electrode 18 configured to treat a target site on or in a patient's body. The housing 12 can be configured as a handpiece, as shown for example in FIG. 1. In other instances, a graspable handpiece is spaced from the housing of the first and the second circuits.

The control unit 34 is configured to provide power to the electrosurgical device 10 for energizing the electrode. As described more fully below, the control unit 34 can be configured to provide energy having a selected waveform and frequency. Some typical control units 34 are configured to provide RF energy to the electrosurgical device 10.

Typically, a cable 32 extends between an electrical connector 33 on the control unit 34 and an electrical connector 31 on the electrosurgical device so as to electrically couple one or more conductive elements on or within the device to one or more corresponding conductive elements of the controller. Some known control units provide three output terminals, with one of the terminals being an energizable terminal for conveying energy, e.g., RF energy, to an energizable element of a handpiece. Such a control unit 34 is usually configured to energize the energizable terminal when a circuit between the two remaining output terminals is completed, as through the closing of a user actuated switch 14.

Some known electrosurgical control units, such as control units manufactured by Ellman International under the brand SURIGTRON and described in U.S. Pat. No. 6,652,514, the contents of which are incorporated herein by reference in their entirety, provide a three-wire output connector for powering and controlling electrosurgical handpieces. Conventional control units can generate, for example, one or more radio-frequency (RF) modulated waveforms, e.g., at a frequency of about 4 mega-Hertz (MHz), which can be delivered to a target site by way of an electrosurgical handpiece having an energizable electrode defining an active surface.

In some cases, the active surface of an electrosurgical system can be configured for non-ablative electrosurgery. As used herein, an ablative procedure is one where the electrode and power settings result in cutting, coagulation, vaporization or other such traumatic disruption to the integrity of treated tissue, and a non-ablative procedure is one where such cutting, coagulation, vaporization or other such traumatic disruption to the integrity of treated tissue does not result.

Some prior electrosurgical systems have incorporated features that attempt to prevent operators from using a worn, non-sterile or otherwise deficient electrosurgical system. For example, U.S. patent application Ser. No. 11/787,245, now U.S. Pat. No. 7,879,032, which is owned by the Assignee of this application, and which is hereby incorporated by reference in its entirety, describes, inter alia, disposable electrosurgical handpieces. A disposable handpiece described in the '032 patent does not accept replacement electrodes and incorporates a battery-voltage detector that renders the handpiece inoperable once the battery's voltage drops below a given threshold voltage. Although such a design improves the safety of handpieces, the battery might discharge regardless of whether the handpiece has actually been used, which can prematurely render the handpiece inoperable, e.g., before its actual useful life has expired.

U.S. patent application Ser. No. 12/455,661, published as U.S. Pub. No. 2010/0312233, which is also owned by the Assignee of this application, and which is hereby incorporated by reference in its entirety, describes, inter alia, shock-free electrosurgical handpieces. Some handpieces described in the '233 Publication have an internal switch that prevents an active electrode surface from being energized unless the surface is in actual contact with a patient's skin. A de-energized electrode surface reduces or eliminates the likelihood that a patient might receive an electrical shock from an electrical arc spanning an air gap between the electrode surface and the patient's skin as the electrode is applied to or removed from the patient's skin.

In some handpieces described in the '233 Publication, arcing can occur inside the handpiece between a portion of the electrode and an energizable element within the handpiece. Either or both of the electrode portion and the energized electrode can degrade (e.g., corrode) over time. Such degradation can increase an electrical resistance between, as well as resistive heating in, these components.

Medical practitioners generally adopt medical devices that provide one or more clinical advantages. Rates of adoption of such devices can be improved if the new devices are backward compatible with existing clinical infrastructure and safe for patients and operators alike. However, known handpieces that have are compatible with existing control units typically have had limited functionality corresponding to the functionality provided by the control unit.

For example, some known control units provide an output connector having three pins, with two pins being signal pins and one of the pins being an energizable pin for energizing an active surface. Closing a circuit between the signal pins causes such a control unit to energize the energizable pin. Such control units generally provide no other functions, e.g., such control units typically lack a processor and output-signal generator that otherwise might allow for two-wire (e.g., serial) communications between the control unit and a device. Consequently, maintaining compatibility with an installed clinical infrastructure has limited the features (e.g., functional capabilities) of handpieces insofar as the installed-base of control units have provided a limited output functionality.

Accordingly, there remains a need for improved electrosurgical systems, including improved disposable handpieces, configured to provide increased functionality while being compatible with existing power supplies and control units. For example, there remains a need for electrosurgical handpieces configured to power a second circuit configured to selectively operate a handpiece accessory using power scavenged from a first circuit configured to energize an electode. In addition, there remains a need for handpieces configured to provide to a user with a cue corresponding to a condition of the handpiece. There also remains a need for handpieces that become unusable in response to a change in condition of the handpiece.

### SUMMARY

The innovations disclosed herein overcome many problems in the prior art and address the aforementioned as well as other needs. The innovations disclosed herein are directed to certain aspects of electrosurgical devices, for example, electrical circuits configured to operate an accessory. In some instances, an accessory can be activated in response to a detected change in a condition. In some embodiments, the accessory is configured to render a used electrosurgical device inoperable upon sensing a change in condition of the device. The change in condition can correspond to a measure of deterioration in device performance. Some disclosed electrosurgical devices can be configured for ablative surgical applications, non-ablative surgical applications, or both.

Some innovative electrosurgical devices are compatible with known control units having a three-wire electrical connector for powering and/or controlling a device. Maintaining compatibility with known control units can allow users to pair a conventional control unit with an innovative electrosurgical device and to use innovative devices without replacing existing clinical infrastructure.

Some innovative devices described herein include an accessory configured to be powered by an electrical current derived from a power source supplied to the electrosurgical device for powering an energizable electrode. For example, an electrosurgical device can include a transformer configured to direct current from a power supply circuit of a conventional control unit to an accessory when the power supply circuit is energized, while simultaneously providing sufficient power to the energizable electrode surface to allow clinical use of the electrode.

Such a transformer can supply a direct current to an accessory circuit. In some instances, the transformer can provide between about 1 Watt (W) and about 5 W, at about 5 Volts (5 VDC), to an accessory while directing a major portion of the supplied power (e.g., about 120 W) to a circuit configured to energize the energizable electrode surface.

Some disclosed electrosurgical devices can have a housing and an electrode defining an energizable surface at least partially positioned externally of the housing. Such a housing can have a first circuit and a second circuit. The first circuit can be configured to selectively direct energy to a power element. The power element can be configured to selectively electrically couple to the electrode. The second circuit can have a selectively operable accessory. The housing can also have a device configured to direct suitable energy from the first circuit to the second circuit to power the second circuit. In some embodiments, the housing is configured as a handpiece.

The accessory can have a condition detector, a controller, and an actuator. The controller can be configured to control the actuator based in part on an output of the detector. In some instances, the actuator can be a relay configured to selectively interrupt the first circuit and thereby to prevent the power element from being energized. In some embodiments, the condition detector is a temperature sensor, a current sensor, a voltage sensor, a timer, or a combination thereof. Such a time can be configured to detect a cumulative duration that the second circuit has operated.

The controller can be configured to selectively bias the relay to selectively restore the first circuit to an uninterrupted state from an interrupted state at least partially in response to an output from the condition detector, and thereby to restore the power element to a selectively energizable state. The second circuit can have a battery and be configured to deliver sufficient power from the battery to the detector when the relay is in the second state as to be able to operate the detector.

In some handpiece embodiments, the electrode has a patient-contact surface and a circuit-contact surface electrically coupled with each other. The electrode can define a non-ablative patient contact surface. The patient-contact surface can be positioned externally of the housing and the circuit-contact surface can be positioned internally of the housing. The power element can define an electrode-contact surface that is selectively electrically couplable to the circuit-contact surface, such that the patient-contact surface is energizable when the power element is energized.

The electrode can be longitudinally movable from an at-rest position to a use position. When the electrode is positioned in the at-rest position, the circuit-contact surface and the electrode-contact surface are so spaced from each other as to electrically decouple the circuit-contact surface from the electrode-contact surface. The circuit-contact surface and the electrode-contact surface can be electrically coupled with each other when the energizable electrode is positioned in the use position.

In some instances, the second circuit is configured to detect whether a condition of the handpiece has surpassed a threshold condition. The second circuit can be configured to render the first circuit at least partially inoperable in response to the detected condition surpassing the threshold condition.

Some handpieces have a power element configured to electrically couple to the electrode. The first circuit can include an energizable element selectively coupleable to and decoupleable from the power element. The second circuit can be configured to decouple the energizable element from the power element so as to render the electrode at least partially inoperable in response to a detected condition surpassing a selected threshold. In some instances, the second circuit is also configured to couple the energizable element to the power element at least partially in response to the second circuit detecting that the condition of the handpiece has not surpassed the threshold.

The threshold condition can correspond to a measure of deterioration in performance of the energizable electrode. For example, the condition can be a temperature of a portion of the electrode, a temperature of a portion of the power-circuit, a duration of electrode operation, a duration of patient contact or a combination thereof.

In some instances, the handpiece accessory has a timer circuit configured to detect a cumulative operating time of the second circuit. The condition can be a cumulative duration that the second circuit has operated and the threshold condition is an upper threshold of the cumulative duration that the second circuit has operated. The cumulative operating time of the second circuit can correspond to a cumulative operating time of the first circuit. In some instances, the cumulative duration that the second circuit has operated can correspond, at least in part, to a cumulative duration that the electrode has operated.

Some disclosed accessories include one or more of a microprocessor, a memory, a light-emitting device, a sound-emitting device, a device configured to interrupt the energy directed to the electrode, an electromechanical device, a sensor configured to detect a condition of the handpiece, a sensor configured to detect an environmental condition external to the handpiece, and combinations thereof.

Innovative methods of operating a handpiece are also disclosed. For example, a portion of a first circuit configured to selectively energize a patient contact surface can be energized. A second circuit configured to operate an accessory can be powered by directing power to the second circuit from the first circuit. A condition can be detected and the accessory can be selectively operated based, in part, on a detected change in the condition. The condition can be a cumulative time of operation of the second circuit.

The accessory can be a relay configured to interrupt the first circuit. The act of selectively operating the accessory based, in part, on a detected change in condition can include biasing the relay to a first state in the absence of a detected change and biasing the relay to a second state in response to a detected change. The relay can be configured to interrupt the first circuit when the relay is in the second state.

The foregoing and other features and advantages will become more apparent from the following detailed description of disclosed embodiments, which proceeds with reference to the accompanying drawings.

### BRIEF DESCRIPTION OF THE DRAWINGS

Unless specified otherwise, the accompanying drawings illustrate aspects of the innovative subject matter described herein.

FIG. 1 illustrates an example of disposable electrosurgical system including an innovative electrosurgical device.

FIG. 2 illustrates a partial cross-sectional view of the innovative electrosurgical device shown in FIG. 1. In FIG. 2, the device is shown in an at-rest configuration.

FIG. 3 illustrates a partial cross-sectional view of the innovative electrosurgical device shown in FIG. 1. In FIG. 3, the device is shown in an activatable configuration.

FIG. 4 shows a schematic representation of an innovative electrosurgical device having an accessory circuit.

FIG. 5 shows a schematic representation of an embodiment of an accessory circuit configured to render an electrosurgical device of the type shown in FIG. 1 inoperable.

FIG. 6 shows a schematic representation of a relay activation circuit that can be incorporated in an innovative handpiece.

FIG. 7 shows a diagram of a method of using an innovative handpiece.

FIG. 8 shows a diagram of a method of biasing a relay in an embodiment of an innovative handpiece.

### DETAILED DESCRIPTION

The following describes various principles related to electrosurgical systems by way of reference to specific examples of electrosurgical handpieces. In some innovative embodiments, a handpiece can be an electrosurgical instrument configured to treat or otherwise manipulate a target site on or in a patient's body.

One or more of the principles can be incorporated in various system configurations to achieve any of a variety of system characteristics. Systems described in relation to particular applications, or uses, are merely examples of systems incorporating the innovative principles disclosed herein and are used to illustrate one or more innovative aspects of the disclosed principles. Accordingly, electrosurgical systems having attributes that are different from those specific examples discussed herein can embody one or more of the innovative principles, and can be used in applications not described herein in detail, for example in ablative surgical applications. Accordingly, such alternative embodiments also fall within the scope of this disclosure.

### Overview

Innovative electrosurgical devices can have an accessory circuit configured to operate one or more accessories and a power circuit configured to direct energy to an energizable electrode. Some innovative devices are configured to direct a portion of the energy from the power circuit to the accessory circuit. Such devices can provide increased device functionality compared to previously known devices, while maintaining compatibility with known control units. For example, some accessory circuits are configured to operate an accessory in response to detecting a change in condition (e.g., a condition of the handpiece or of an operating environment). Some innovative accessory circuits are also configured to confirm that such a change in condition has occurred. Related electrosurgical systems are also described.

As noted above, electrosurgical devices disclosed herein can be configured for non-ablative electrosurgery. In some instances, such electrical devices are configured to prevent traumatic disruption to a tissue as well as to keep any tissue disruption below a patient's pain threshold. For example, some disclosed electrical devices are configured to deliver energy to a patient's skin without the need for anesthetizing the patient. Although difficult to quantify the precise limits of such power thresholds, applying an energy flux of 4,000 Watts per square centimeter (W/cm²) for about one second (1 s) probably would not ablate skin tissue, but might cause necrosis of some tissue. On the other hand, it is presently believed that an energy flux of about 2,000 W/cm² applied for between about 2 and about 3 s can be applied to skin tissue to obtain desirable clinical outcomes. Lower flux levels can be applied for longer times, and higher flux levels might be applied for shorter times, without damaging tissues.

An innovative device 10 (shown in FIG. 1) can have a housing 12 with an energizable electrode element extending from a distal end of the housing. A three-pin connector 31 can be positioned adjacent a proximal end of the housing. A cable 32 can extend between and electrically couple the device 10 and the control unit 34. In this embodiment, the housing 12 serves as a handpiece. In other embodiments, the housing could be, for example, a shaft positioned between an electrode and a handpiece.

As used herein, a “handpiece” means an instrument configured such that a user can hold it in his hand during use. Usually, a handpiece is spaced from an instrument portion (e.g., an energizable patient contact surface) configured to be used on or inserted into a patient's body. Hereinafter, a handpiece will be used as a representative embodiment of a housing 12.

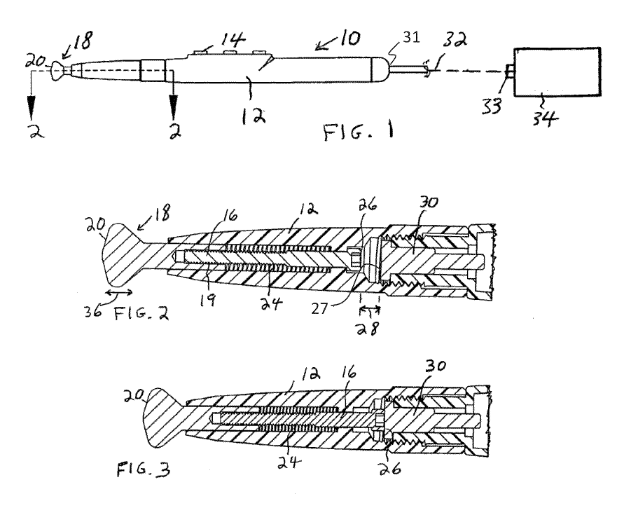

The electrode 18 can be longitudinally movable to open and close a gap 28 between an electrically conductive power element 30 and the electrode forming a mechanical switch within the handpiece. FIG. 2 shows the gap 28 in an open position and FIG. 3 shows the gap in a closed position.

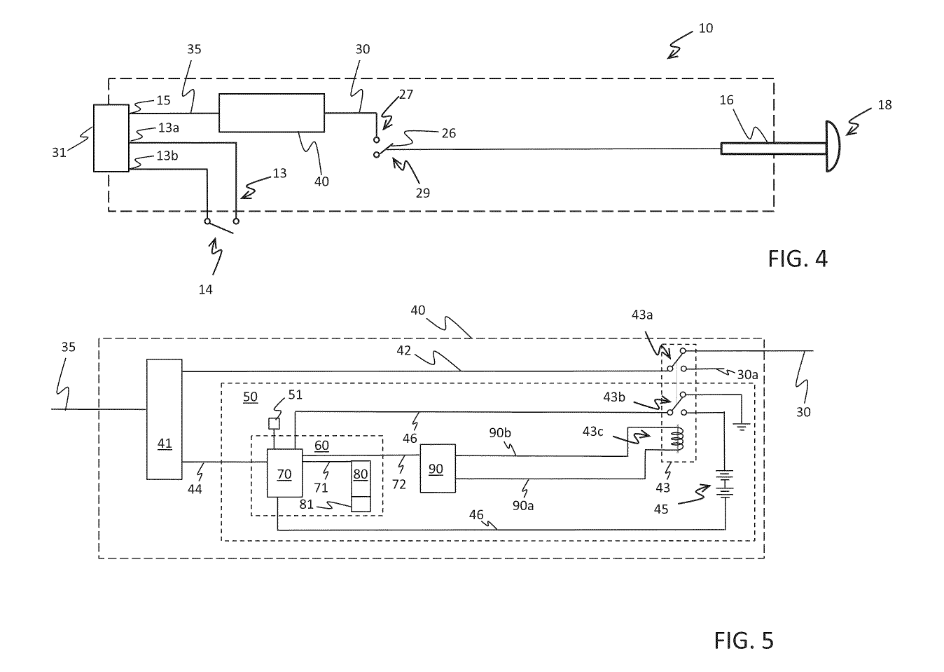

As shown in FIG. 4, some innovative handpieces include an accessory circuit 40. The circuit 40 can be configured to operate a variety of accessories. In some instances, the accessory circuit can be configured to render the handpiece 10 inoperable in response to detecting a change in a condition. As shown in FIG. 5, the accessory circuit can be configured to confirm, subsequent to rendering the handpiece inoperable, that such a change in condition did occur. In some instances, an accessory circuit can also be configured to render the handpiece operable if the change in condition did not occur. In other embodiments, the accessory circuit can be configured to confirm that a change in condition occurred before the circuit renders the handpiece inoperable.

In the particular embodiment shown in FIG. 5, the accessory circuit 40 is configured to switch a relay 43 from a first, operable state to a second, inoperable state when a timer reaches a threshold value of time corresponding at least in part to a duration that the electrode 18 has been energized. In this example, the accessory circuit 40 is configured to confirm that the threshold value of time has been surpassed if the relay 43 is in the second, inoperable state. For example, when the relay 43 is in the second, inoperable state, a battery-powered portion of the accessory circuit 40 can be activated and the value of the timer can be compared to the threshold value of time. The accessory circuit 40 can be configured such that if the value of the timer is less than the threshold value, the accessory circuit switches the relay from the second, inoperable state to the first, operable state, and if the value of timer is greater than the threshold value, the relay remains in the second, inoperable state.

Such confirmation of the detected change in condition can be useful to prevent rendering the handpiece inoperable prematurely, which might happen since a latching relay can sometimes switch states without being provided with an activation current. For example, inertial forces within an electromechanical relay can cause the relay to switch states if the relay undergoes a rapid acceleration or deceleration. In practice, an electromechanical relay might be switched from one state to another if a handpiece is subjected to a sufficient mechanical impact, such as if it is dropped on a hard floor. A solid-state latching relay can also switch between states without being provided an activation current. If such an inadvertent switching occurs, the battery operated portion of the circuit 40 can bias the relay to the first operable state while the handpiece remains usable.

### Handpieces

FIG. 1 shows a schematic view of one possible example of an innovative electrosurgical device 10 having a housing 12, an energizable electrode 18 extending from a distal end of the device and a cable 32 extending from a connector 31 positioned adjacent a proximal end of the device. The electrode 18 can define a non-ablative contact surface 20. The cable 32 can extend between the connector 31 on the device and a connector 33 on a control unit 34 to deliver power to the device. In FIG. 1, the housing 12 is configured as a handpiece. In other possible embodiments, the housing 12 can be spaced from the graspable handpiece.

As the partial cross-sectional view in FIG. 2 shows, the energizable electrode 18 can have a longitudinally oriented and electrically conductive shank 16 extending proximally inside the housing 12. The handpiece 10 can also have a proximally positioned power element 30 that can be electrically coupled with an energizable power source (e.g., a power pin 15 of the proximally positioned connector 31 coupling the cable 32 to the device 10).

The illustrated electrode 18 defines an internally threaded bore 19 that threadably engages a correspondingly threaded external surface of the shank 16. In other embodiments, the shank 16 and the electrode can form a unitary construction. The elongated electrically-conductive shank 16 and the electrode 18 can together move longitudinally of the handle 12 between a distal position (FIG. 2) and a proximal position (e.g., FIG. 3). As shown in FIG. 2, the electrode 18 can be outwardly biased by an internally positioned compression spring 24 such that the electrode is biased toward the distal, at-rest position.

When displaced from the at-rest position shown in FIG. 2, the electrode can return to the distal position under the biasing force of the spring 24. In the at-rest, distal position, the circuit-contact surface 26 of the shank 16 and the electrode-contact surface 27 of the power element 30 are so spaced from each other as to form a gap 28 and electrically decouple the circuit-contact surface from the electrode-contact surface (e.g., the gap 28 is sized such that a voltage potential between the surfaces 26, 27 is insufficient to cause arcing between the surfaces).

The electrode 18 can be urged proximally, as by contact between the surface 20 of the electrode 18 and a patient's skin, toward a proximal position in which the gap 28 between the proximal end of the shank 16 and the distal end of the power element 30 is closed, as shown in FIG. 3. In such a proximal position, the circuit-contact surface 26 of the shank 16 can urge against the electrode contact surface 27 of the power element 30. Upon releasing a proximally directed longitudinal force applied to the electrode 18, the electrode can return to the at-rest position under the biasing force of the spring.

The 3-pin connector 31 is shown schematically in FIG. 4. An externally positioned and user-operable switch 14 can be configured to close a circuit between two of the three connector pins (e.g., pins 13a, 13b). When the switch 14 closes, a control circuit 13 between the pins 13a, 13b is closed, signaling the control unit 34 to energize the connector 33 (FIG. 1) and, correspondingly, the pin 15 (FIG. 4). In some instances, the switch 14 is positioned on or in the medical device. In other instances, the switch 14 is spaced from the device. For example, the switch 14 can be configured as a foot-operable switch that is spaced apart and physically decoupled from the device 10. The power pin 15 is electrically coupled to a power supply portion 35 of the circuit that energizes the electrode 18.

As shown in FIG. 4, the power supply circuit can be configured to selectively direct energy to the electrode 18 and thereby to selectively energize the energizable surface 20. For example, the power circuit can include an electrode switch 29 (corresponding to the gap 28 positioned internally of the housing 10 and having a first (e.g., closed) state in which the energizable electrode is electrically coupled to the third pin 15 and a second (e.g., open) state in which the energizable electrode is electrically isolated from the third pin). When the electrode 18 is in the operable proximal, position, the switch 29 (air gap 28) closes and the handpiece 10 can be placed in an operable state, allowing the energizable electrode to be energized (e.g., provided that the user-operable switch 14 is actuated so the control unit 34 energizes the power pin 15 of the proximally positioned connector 31).

The handpiece 12 can be electrically-insulating and can have a user-operable switch 14 configured to close a control circuit 13 (FIG. 4) for initiating operation of the control unit 34 (FIG. 1). When the power element 30 is energized, arcing can occur between the circuit-contact surface 26 and the electrode-contact surface 27 as the electrode 18 is moved in a proximal direction. After a number of cycles of making and breaking contact between the circuit-contact surface 26 and the electrode-contact surface 27, such arcing can degrade (e.g., corrode) either or both of the surfaces and locally increase an electrical resistance through the electrical coupling between the power element 30 and the energizable electrode 18. In addition, some handpiece users vary the pressure applied between the patient contact surface 20 and the patient's skin, intermittently forming gaps between the circuit-contact surface 26 and the electrode-contact surface 27. Such intermittently formed gaps may promote arcing between the circuit-contact surface 26 from the electrode-contact surface 27. After some time, such arcing can degrade either or both of the surfaces. An accessory circuit, as described below, can render the handpiece inoperable or provide the user with another cue, for example, when the surfaces have been degraded.

The movable electrode shank 16 (FIG. 1) with its corresponding circuit contact surface 26 and the power element 30 with its corresponding electrode-contact surface 27 are shown schematically in FIG. 4 as elements of a switch 29. As described above, when the gap 28 (FIG. 1) between the circuit contact surface 26 and the electrode-contact surface 27 closes (e.g., when the switch 29 closes), the electrode 18 can be energized from the energized pin 15.

### Accessories and Accessory Circuits

As indicated in FIG. 4, the innovative handpiece 10 can include an accessory circuit 40 configured to operate any of a variety of device accessories. As used herein, “accessory” means a component or a subsystem configured to operate independently of or as an adjunct to the energizable electrode.

The accessory circuit 40 can receive power from a power supply portion 35 of the circuit that provides power to the energizable electrode 18. Such an accessory configuration can provide the handpiece 10 with improved functionality compared to conventional handpieces and backward compatibility to conventional control units 34 (FIG. 1). In some embodiments, the accessory circuit 40 includes a control circuit configured to render the handpiece 10 inoperable in response to detecting that a condition has surpassed a threshold condition.

For example, a control circuit can be configured to remove an otherwise energizable active electrode surface from a power supply circuit after the active electrode surface has been energized and de-energized a selected number of times. As another example, a control circuit can be configured to remove the electrode surface from a power supply circuit based, at least in part, on a cumulative time that the electrode surface has been energized.

As an example, an innovative a handpiece 10 is configured to render the energizable electrode 18 inoperable after a predetermined duration of use of the handpiece. When the relay 43 is in a first, operable state (shown in FIG. 5), the circuit that powers the electrode remains closed (e.g., the electrical coupling 42 is coupled to the power element 30) and the electrode 18 can be selectively operated by a user. When the relay 43 is in a second, inoperable state (e.g., the relay terminal 43a couples the coupling 42 to the stub 30a), the circuit that powers the electrode is left open and the electrode 18 is inoperable by a user.

As FIG. 4 shows, the handpiece 10 includes an accessory circuit 40 that derives power from a circuit that powers the electrode 18. The power supply portion 35 of the power circuit in the handpiece 10 can be electrically coupled to a transformer 41 (e.g., a current sense transformer). An energizable element 42 coupled to the transformer can be coupled to the relay terminal 43a. One of the relay outputs can be configured as a circuit stub 30a. The other relay output can be electrically coupled to the power element 30 defining the electrode-contact surface 27 (FIGS. 2 and 4).

The transformer 41 shown in FIG. 5 can have a second output 44 configured to power an accessory. In the illustrated embodiment, the accessory is a circuit 50 configured to interrupt the circuit configured to supply power to the electrode 18 by switching the relay terminal 43a from the first state, shown in FIG. 4 (e.g., electrically coupled to the power element 30), to the second state (e.g., electrically coupled to the stub 30a). In some embodiments, the circuit 50 is configured to switch the relay terminal 43a from the first state to the second state in response to detecting that a condition has surpassed a threshold condition. The relay 43 can be an independent device apart from the user-operable switch 14 and the internal electrode switch 29.

For example, the illustrated circuit 50 has a clock 51 coupled to a computing device 60 having a processor 70 and a memory 80. The processor 70 and memory 80 are coupled to each other by a bus 71. In some embodiments, the computing device 60 and the clock 51 are integrated into a single electronic component. One example of such an integrated component is a commercially available semiconductor device, such as a Microchip PIC18F1320.

The clock 51, processor 70 and memory 80 can be configured as a timer configured to monitor a duration that the computing device 60 and clock 51 have been powered. With the accessory 40 shown in FIG. 5, the duration that the computing device 60 and clock 51 have been powered can correspond to a duration that the electrode 18 has been powered, since the accessory circuit 40 derives power from the same source as the electrode 18 (e.g., power supply portion 35).

An upper-threshold duration of electrode operation can be stored in the memory 81 (shown, for example, as being a register in memory 80). If the duration that the computing device 60 and clock 51 has operated exceeds the threshold duration stored in the memory 81, the computing device 60 can transmit a signal across the bus 72 to a relay activation circuit 90 for biasing the relay 43 to a desired state.

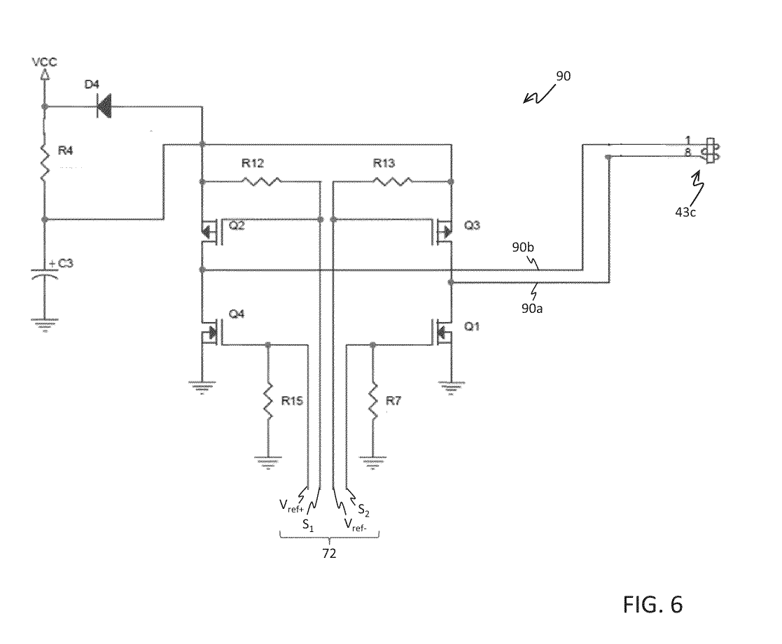

The relay activation circuit 90 (FIG. 6) can be configured to provide an activation current to the relay 43 corresponding to one or more output signals from the processor 70. For example, the circuit 90 can provide a current having a first polarity (i.e., the current can pass from the coupling 90a to the coupling 90b) corresponding to a first signal S₁ from the computing device 60 or a second polarity (i.e., the current can pass from the coupling 90b to the coupling 90a) corresponding to a second signal S₂ from the computing device 60. The relay 43 can be configured to bias toward a first state (e.g., shown in FIG. 5) in response to the first polarity and to bias toward a second state (e.g., relay terminal 43a coupled to stub 30a) in response to the second polarity. The activation circuit 90 includes a plurality of transistors (Q1, Q2, Q3 and Q4) arranged such that a single capacitor C3 can be used to provide a short-duration (e.g., between about 5 ms and about 10 ms) pulse of an activation current to the relay with a desired polarity.

Even in the absence of an activation current that would bias the relay 43 toward the second state, some latching relays can switch to the second state prematurely and render the electrode 18 inoperable. As one approach for ensuring that the relay 43 remains in, or is switched to, a desired state, the accessory circuit 40 can be configured to provide a biasing current having a desired polarity to the relay 43 from time to time. For example, when the relay 43 is in the first state, shown in FIG. 5, the computing device 60 can transmit a signal to the relay activation circuit 90 to initiate an activation current. As noted above, the polarity of the activation current can correspond to an intended state of the relay, based, at least in part, on whether a threshold condition has been surpassed. For example, while the cumulative time of operation of the circuit 40 is less than an upper threshold time, the polarity of the activation current can be selected to bias the relay to the state shown in FIG. 5 (e.g., to maintain an electrical coupling between the electrode 18 and the energizable pin 15 of the connector 31). When the cumulative time of operation of the circuit exceeds the upper threshold time, the polarity of the activation current can be selected to bias the relay to a second state to render the handpiece inoperable.

FIGS. 5 and 6 show a circuit (e.g., circuit 50 and activation circuit 90) configured to bias the relay 43 to a desired state irrespective of the relay's present state. For example, if the relay 43 is in the state shown in FIG. 5, the circuit 50 can receive power from the transformer 41 and the relay 43 can be biased to the first state or the second state as described above.

On the other hand, when relay 43 is in the second state (e.g., the relay terminal 43a is coupled to the stub 30a), the relay terminal 43b is electrically coupled to a battery 45, since the respective states of the terminals 43a and 43b of the illustrated relay correspond to each other (i.e., when the terminal 43a is coupled to the stub 30a, the terminal 43b is coupled to the battery, and when the terminal 43a is coupled to the power element 30, the terminal 43b is coupled to ground). With such a configuration, the circuit 50 can be powered and operable regardless of the state of the relay 43, and the relay 43 can be biased to a desired state by the timer and activation circuit 90. With a circuit configuration as shown in FIG. 5, even an unintended switch of the relay 43 (as by dropping the handpiece) can be corrected, since the relay can receive a biasing current corresponding to a desired state regardless of the relay's state.

### Methods for Operating an Innovative Handpiece

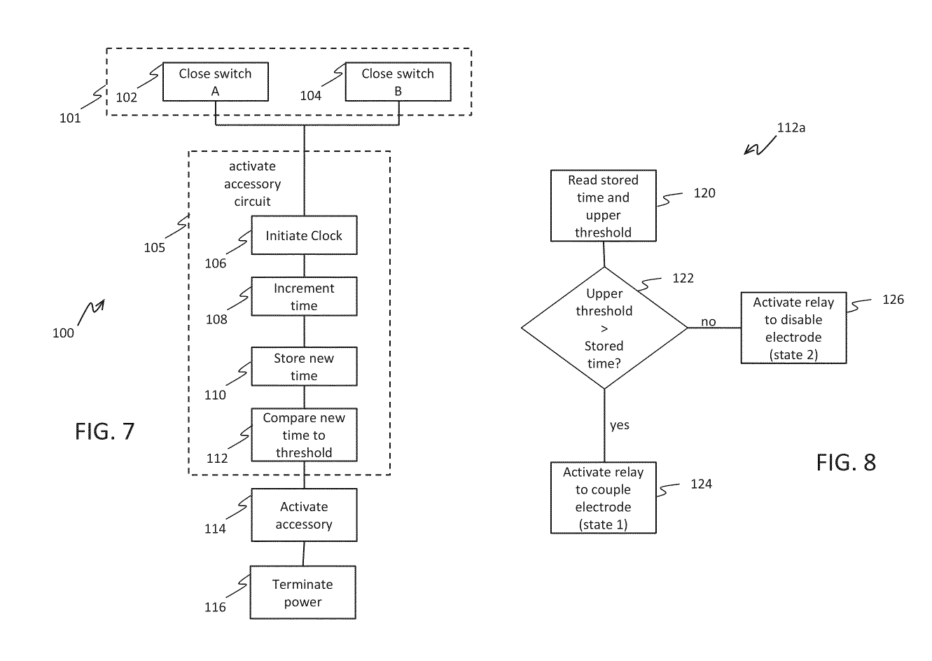

Methods for operating an embodiment of an innovative handpiece 10 having an accessory circuit are shown in FIG. 7. For example, operation of the circuits shown in FIG. 5 will be described with reference to the diagram 100 shown in FIG. 7 and the diagram 112a shown in FIG. 8. As described above, the handpiece 10 can have a user operable switch 14 configured to activate a control unit for supplying power to the handpiece 10. In a first method act 101, a user can attempt to energize the electrode 18 of an innovative handpiece 10, as by closing the switch 14 (102) and urging the electrode against a patient's skin to close the switch 29 (104). If the relay terminal 43a is in the state shown in FIG. 5, the transformer 41 will direct power to the accessory circuit 40, activating the accessory circuit (105). When the accessory circuit 40 is powered, the clock 51 is initiated (106) and a time (n) is incremented (108). The processor 70 can write the new time (n+δt) to the memory 80 (110) and the processor 70 can compare the new time (n+δt) to, for example, an upper threshold time (112). When the new time (e.g., a cumulative operating time) reaches a threshold (e.g., some percentage (x %) of an upper threshold time), a handpiece accessory can be activated (114). The clock 51 can continue to increment the time until the accumulated new time reaches an upper threshold time, after which, the power supply can be terminated (116).

The accessory activated at 114 can be any of a variety of accessories. For example, the handpiece can include one or more light emitting diodes (LEDs) that the circuit 40 can activate to alert a user that the handpiece has been operated for a given duration (e.g., x % of an upper threshold time). Other examples of accessories that can be activated at 114 are described below.

FIG. 8 shows an example comparison 112a in which the accessory is the relay 43 shown in FIG. 5. As FIG. 8 shows, the comparison act 112 can include reading a stored time and an upper threshold time (120) and determining whether the upper threshold time exceeds the stored time (122). If the upper threshold time exceeds the stored time, the relay 43 can be biased (e.g., as described above) to a state in which the electrode 18 is or remains energizable (124). Alternatively, the relay 43 can be biased to a state in which the electrode is not energizable (126), rendering the handpiece 10 inoperable.

### Other Exemplary Embodiments

The accessory circuits and methods described above generally concern activating a handpiece accessory in response to detecting a predetermined condition. In FIGS. 5, 6 and 7, the illustrated circuits and methods pertain, in part, to detecting a cumulative time that an accessory circuit has operated. With some accessory circuits, the cumulative circuit operating time generally corresponds to a cumulative time that the electrode 18 has been supplied with power, and can correspond to a degree of electrode deterioration when the cumulative time that the electrode has been supplied with power corresponds to a degree of electrode deterioration.

Nonetheless, a degree of electrode deterioration can correspond to a variety of observable conditions. Moreover, other conditions unrelated to electrode deterioration can be observed and used as the basis for activating an accessory.

Generally, an accessory circuit can include a condition detector configured to detect any of a variety of conditions, regardless of whether the respective conditions are related to electrode deterioration. For example, observable conditions can include a cumulative number of times that an electrode has been energized and/or de-energized, an electrode temperature, an environmental temperature (e.g., a temperature of a patient's skin, or an air temperature), a temperature of a handpiece component (e.g, adjacent the internal spark gap 28), a cumulative time that an electrode has been powered, a duration that an electrode has been powered continuously, a characteristic of the power supplied to the electrode (e.g., power, voltage, current, impedance), a degree of current sinking of a neutral plate, a ratio of power output by the electrode to power at such a neutral plate, a circuit impedance, a supply signal provided to a control unit, and combinations thereof.

As well, accessory circuits can be configured to activate a wide variety of accessories in response to an observed condition surpassing a threshold. For example, such an accessory can include a relay, such as a relay configured to interrupt power deliver to the electrode; a visual cue, such as an LED; an audible cue, for example, an audible alarm; a digital display, such as a display showing a measured value of the observed condition).

As an example, an accessory circuit can include a processor configured to monitor one or more inputs to the handpiece or to monitor one or more external sensors. An accessory circuit can power an LED, and the accessory circuit (e.g., the processor) can be configured to operate the LED to provide a visual cue indicating a status of the handpiece (e.g., constant illumination can indicate that the electrode is energized, intermittent illumination can indicate the remaining useful life of the electrode, another LED can indicate the duration that the electrode has been active in a given treatment session, etc.). The accessory circuit can be configured to provide an audible tone to indicate a status of the handpiece. As yet another example, the accessory circuit can be configured to provide a signal to a control unit to adjust the amount of power supplied to the handpiece upon detecting a given condition.

Although accessory circuits described in detail above incorporate a relay configured to interrupt a power supply to the energizable electrode 18, other approaches for interrupting the power supply circuit can be incorporated in accessory circuits. For example, a thermal fuse can be incorporated into the accessory circuit and power supplied to the electrode can be routed (or re-routed in response to a sensed condition) through the thermal fuse. A heating element positioned adjacent the thermal fuse can heat the fuse until it fails and opens the power supply circuit, rendering the electrode inoperable. Alternatively, a motor can be actuated in response to a sensed condition to move a contact, interrupting a power supply to the electrode. A solenoid can be activated to move such a contact.

This disclosure references the accompanying drawings, which form a part hereof, wherein like numerals designate like parts throughout. The drawings illustrate specific embodiments, but other embodiments may be formed and structural and logical changes may be made without departing from the intended scope of this disclosure.

Directions and references (e.g., up, down, top, bottom, left, right, rearward, forward, etc.) may be used to facilitate discussion of the drawings but are not intended to be limiting. For example, certain terms may be used such as “up,” “down,”, “upper,” “lower,” “horizontal,” “vertical,” “left,” “right,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” surface can become a “lower” surface simply by turning the object over. Nevertheless, it is still the same surface and the object remains the same. As used herein, “and/or” means “and” or “or”, as well as “and” and “or.”

Incorporating the principles disclosed herein, it is possible to provide a wide variety of systems configured to render an electrosurgical handpiece inoperable at or near an end of the handpiece's safe useful life, in addition to the systems described above.

The technologies from any example can be combined with the technologies described in any one or more of the other examples. Accordingly, this detailed description shall not be construed in a limiting sense, and following a review of this disclosure, those of ordinary skill in the art will appreciate the wide variety of electrosurgical systems that can be devised using the various concepts described herein.

Moreover, those of ordinary skill in the art will appreciate that the exemplary embodiments disclosed herein can be adapted to various configurations without departing from the disclosed principles. Thus, in view of the many possible embodiments to which the disclosed principles can be applied, it should be recognized that the above-described embodiments are only examples and should not be taken as limiting in scope. We therefore reserve all rights to the subject matter disclosed herein, including the right to claim all that comes within the scope and spirit of the following claims, as well as all aspects of any innovation shown or described herein.

1. An electrosurgical device comprising:
a housing and an electrode defining an energizable surface at least partially positioned externally of the housing;
a first circuit configured to selectively direct energy to a power element configured to selectively electrically couple to the electrode;
a second circuit comprising a selectively operable handpiece accessory; and
a device configured to direct energy from the first circuit to the second circuit, wherein the energy directed to the second circuit is suitable for powering the second circuit.

2. The device of claim 1, wherein:
the electrode comprises a patient-contact surface and a circuit-contact surface electrically coupled with each other, wherein the patient-contact surface is positioned externally of the housing and the circuit-contact surface is positioned internally of the housing; and
the power element comprises an electrode-contact surface, wherein the circuit-contact surface and the electrode-contact surface are selectively electrically couplable to each other such that the patient-contact surface is energizable when the power element is energized.

3. The device of claim 1, wherein the housing comprises a handpiece.

4. The device of claim 3, wherein the electrosurgical device comprises a non-ablative device configured to perform a cosmetic procedure, wherein the electrode defines a non-ablative patient contact surface.

5. The device of claim 2, wherein the electrode is longitudinally movable from an at-rest position to a use position, wherein, when the electrode is positioned in the at-rest position, the circuit-contact surface and the electrode-contact surface are so spaced from each other as to electrically decouple the circuit-contact surface from the electrode-contact surface.

6. The device of claim 5, wherein the circuit-contact surface and the electrode-contact surface are electrically coupled with each other when the energizable electrode is positioned in the use position.

7. The device of claim 1, wherein the second circuit is configured to detect whether a condition of the handpiece has surpassed a threshold condition.

8. The device of claim 7, wherein the second circuit is further configured to render the first circuit at least partially inoperable in response to the detected condition surpassing the threshold condition.

9. The device of claim 1, further comprising a power element configured to electrically couple to the electrode, wherein the first circuit comprises an energizable element selectively coupleable to and decoupleable from the power element, and wherein the second circuit is configured to detect whether a condition of the handpiece has surpassed a selected threshold condition and to decouple the energizable element from the power element so as to render the electrode at least partially inoperable in response to the condition surpassing the selected threshold.

10. The device of claim 9, wherein the second circuit is further configured to couple the energizable element to the power element at least partially in response to the second circuit detecting that the condition of the handpiece has not surpassed the threshold.

11. The device of claim 7, wherein the threshold condition corresponds to a measure of deterioration in performance or degree of use of the energizable electrode.

12. The device of claim 7, wherein the condition of the energizable electrode comprises a temperature of a portion of the electrode, a temperature of a portion of the power-circuit, a duration of electrode operation, a duration of patient contact or a combination thereof.

13. The device of claim 9, wherein the condition comprises a cumulative duration that the second circuit has operated and the threshold condition comprises an upper threshold of the cumulative duration that the second circuit has operated.

14. The device of claim 13, wherein the cumulative duration that the second circuit has operated corresponds, at least in part, to a cumulative duration that the electrode has operated.

15. The device of claim 1, wherein the accessory comprises a microprocessor, a memory, a light-emitting device, a sound-emitting device, a device configured to interrupt the energy directed to the electrode, an electromechanical device, a sensor configured to detect a condition of the handpiece, a sensor configured to detect an environmental condition external to the handpiece, or a combination thereof.

16. The device of claim 1, wherein the electrode defines a non-ablative patient-contact surface.

17. The device of claim 1, wherein the accessory comprises a timer circuit configured to detect a cumulative operating time of the second circuit.

18. The device of claim 17, wherein the cumulative operating time of the second circuit corresponds to a cumulative operating time of the first circuit.

19. The device of claim 1, wherein the device accessory comprises a condition detector, a controller, and an actuator, wherein the controller is configured to control the actuator based in part on an output of the detector.

20. The device of claim 19, wherein the actuator comprises a relay configured to selectively interrupt the first circuit and thereby to prevent the power element from being energized.

21. The device of claim 20, wherein the condition detector comprises a temperature sensor, a current sensor, a voltage sensor, a timer, or a combination thereof.

22. The device of claim 20, wherein the condition detector comprises a timer configured to detect a cumulative duration that the second circuit has operated.

23. The device of claim 20, wherein the controller is configured to selectively bias the relay to selectively restore the first circuit to an uninterrupted state from an interrupted state at least partially in response to an output from the condition detector, and thereby to restore the power element to a selectively energizable state.

24. The device of claim 23, wherein the second circuit further comprises a battery, wherein the second circuit is configured to deliver sufficient power from the battery to the detector when the relay is in the second state as to be able to operate the detector.

25. A method of operating an accessory of an electrosurgical device, the method comprising:
energizing a portion of a first circuit configured to selectively energize a patient contact surface;
powering a second circuit configured to operate the accessory by directing power to the second circuit from the first circuit.

26. The method claim 25, further comprising detecting a condition and selectively operating the accessory based, in part, on a detected change in the condition.

27. The method of claim 26, wherein the condition comprises a cumulative time of operation of the second circuit.

28. A method of manufacturing an electrosurgical device, the method comprising:
positioning an electrode defining an energizable surface at least partially externally of a housing;
configuring within the housing a first circuit to selectively electrically couple to a power element being selectively electrically couplable to the electrode;
configuring within the housing a device to direct to a second circuit a portion of energy provided to the first circuit, wherein the second circuit comprising a selectively operable accessory.

29. The method of claim 28, wherein the accessory comprises a condition detector, a controller, and an actuator, wherein the method further comprises configuring the controller to control the actuator based in part on an output of the detector.

30. The method of claim 29, wherein the actuator comprises a relay, the method further comprising configuring the relay to selectively interrupt the first circuit so as to prevent the power element from being energized.

31. The method of claim 29, wherein the condition detector comprises a temperature sensor, a current sensor, a voltage sensor, a timer, or a combination thereof.

32. The method of claim 30, further comprising configuring the controller to selectively bias the relay to selectively restore the first circuit to an uninterrupted state from an interrupted state at least partially in response to an output from the condition detector.

33. The device of claim 23, wherein the second circuit further comprises a battery, wherein the second circuit is configured to deliver sufficient power from the battery to the detector when the relay is in the second state as to be able to operate the detector.
